(19) United States
(12) Patent Application Publication
(10) Pub. No.: US 2018/0223236 A1
(43) Pub. Date: Aug. 9, 2018

(54) HORIZONTALLY INCLINED TROUGH REACTOR AND USES THEREFOR

(71) Applicant: DIESELPRIME LTD, Gdeide-Maker (IL)

(72) Inventors: MUHAMMAD KAIYAL, Jdeide-Maker (IL); WALID BADRAN, Kabul (IL)

(73) Assignee: DIESELPRIME LTD, Gdeide-Maker (IL)

(21) Appl. No.: 15/747,777

(22) PCT Filed: Jul. 30, 2016
(86) PCT No.: PCT/IB16/54599

§ 371 Date: Jan. 26, 2018

Related U.S. Application Data
(60) Provisional application No. 62/199,235, filed on Jul. 31, 2015.

(51) Int. Cl. C12M 1/12; C10L 1/02; B01J 14/00; C12P 7/64; C12M 1/00

(52) U.S. Cl. CPC C12M 23/04; C10L 1/026; B01J 14/005; C12P 7/649; C12M 29/04; C10L 2290/26; C12M 29/06; C12M 27/18; C12M 47/10; C12M 21/12

(57) ABSTRACT

A trough reactor, including an elongated trough shaped enclosure, a stock feed inlet, a reactant feed-in and distribution system, at least one separation plate extending downwardly underneath the elongated trough shaped enclosure and at least one separation column forming a continuous gravitational decanter with at least one outlet and a posterior outlet, as well as a method of catalyzing a reaction in a trough reactor, including horizontally inclining an elongated trough shaped enclosure, supplying a feed of substrate into the elongated trough shaped enclosure, supplying and distributing a feed-in of a reactant into the elongated trough shaped enclosure; disposing a separation plate essentially vertically in the elongated trough shaped enclosure, substantially obstructing a spontaneous gravitational flow along the elongated trough shaped enclosure by the separation plate, providing a separation column of an essentially hollow vertical structure, separating the substrate or reactant or a product by a means of continuous gravitational decantation process, draining from an outlet at a bottom portion of the separation column an excessive portion of the substrate or reactant or a product and draining a portion of the substrate or reactant or a product from a posterior outlet, are described.

### TECHNICAL FIELD

In general, the present invention pertains to the art of chemical engineering. In particular, the invention relates to a horizontally inclined trough shaped reactor employing a gravitational flow as well as to various chemical reactions catalyzed therein,

### BACKGROUND ART

It is believed that the current state of the art is represented by the following patent literature: US2013143313, US20140024109, US2004186307, US20110281339, U.S. Pat. No. 8,658,420, U.S. Pat. No. 8,404,005 and CA1149726. US2013143313 which is believed to be the closest prior art discloses a harvesting device for capturing a biological product directly by binding the secreted biological product with a resin, discarding the nutrient medium and eluting the biological product as a concentrated solution, eliminating the steps of sterile filtration and volume reduction, thus allowing one to combine the steps of recombinant expression and separation of a biological product. The method of US2013143313 allows loading of resin for column-purification, eliminating all steps of perfusion process and maintaining a sink condition of a toxic product in nutrient medium to optimize productivity of host cells. US2013143313 allows harvesting of solubilized inclusion bodies after the cells have been lysed and refolding of proteins inside the bioreactor.

US20140024109 discloses a composting system is provided that uses gravity and natural thermal convection to yield a compact, modular, plug-flow compost reactor requiring minimal aeration and agitation energy. The compost reaction of US20140024109 takes place in a self-supporting containment unit which is mounted at an angle with respect to its supporting base pad such that minimal external energy is required to mix and transport the composting material during its residence time within the container. The system of US20140024109 uses natural convection to supplement external energy in the introduction of air into and through the material. Furthermore, the configuration of the containment unit in US20140024109 and its supporting structures allow rapid deployment of compost facilities with minimal permanent civil work and minimal space requirements in a manner that enables subsequent relocation of the equipment.

US2004186307 discloses a method of producing fuel from vegetable or animal fat having a free fatty acid content by means of catalytic esterification reactions. The method includes esterification of free fatty acids at a higher temperature in a vacuum with one or more multivalent alcohols accompanied by solid neutral catalysts, which are present in a packing bed inside a reactor, whereby the fat travels from top to bottom in the reactor with the alcohol running counter current and a mixture containing alcohol and water being removed from an upper part of the reactor by means of a vacuum effect. US2004186307 discloses the apparatus for implementing the methods.

### SUMMARY OF THE INVENTION

There is provided in accordance with some embodiments of the present invention a trough reactor comprising an elongated trough shaped enclosure horizontally inclined at an angle sustaining a spontaneous gravitational flow, a stock feed inlet suppling substrate into the trough reactor, a reactant feed-in and distribution system supplying and distributing a reactant in the trough reactor, at least one separation plate extending downwardly underneath a bottom face of the elongated trough shaped enclosure, at least one separation column forming a continuous gravitational decanter with at least one outlet and a posterior outlet.

The trough reactor, in accordance with some preferred embodiments of the present invention, is implemented inter alia for the production of biodiesel. State-of-the-art techniques of biodiesel fuel production often suffer from the several drawbacks. Firstly the state-of-the-art techniques required adding a substantial portion of water or aqueous buffer to the reaction, which have increased the percentage of free fatty acids in reaction product the and hence required a polishing process, such a caustic wash, to decrease the percentage of free fatty acid (FFA) in the fuel product resulting an emulsion, separated typically by centrifugation to remove the resulting soap, which have increased the overall cost of the process and reduced the yields thereof.

Moreover, state-of-the-art techniques of biodiesel fuel production that required adding water or aqueous buffer to the reaction has contributed to the dissolution of the enzyme from the matrix it was immobilized to, resulting in leakage of the enzyme from the matrix, reducing the effective concentration of the enzyme over time.

In accordance with state-of-the-art methods the amount of reactant (alcohol) added to the reaction system was imitated. Since the enzyme loses its activity in the environment which is rich in alcohol. According to current technology a series of consecutive tower or column reactors was required to split the amount of the distributed alcohol, which required more equipment, more control and increased dramatically the setup costs.

In state-of-the-art techniques there were limitations on the type of oils to be used. In systems designed to operate at relatively low temperatures, no solid fats or oils, such as palm oil, which become liquid only at temperatures above 35 degrees, were applicable. Only after premixing the solid fats or oils with other lighter oils the substrate could have been used as stock feed-in, which once again complicated and increased the cost of the process.

Furthermore, state-of-the-art systems have not provided for an easy scaling up or down of the production plant. For example, to increase or decrease the output, it is necessary to build a new unit or remove an existing one. Finally, enzyme reactivation difficulties are characteristic of the prior art systems, for an enzyme that underwent even moderate deactivation, for instance to 90% instead of 97% converse rate (turnover number). There was no easy process to easily re-activate the enzyme in situ, forcing to occasionally empty the entire batch of enzyme to re-activate it off-site, thereby making the process less continuous and jeopardizing the enzyme especially in large quantities.

The trough reactor, in accordance with various embodiments of the present invention, implemented for production of biodiesel, overcomes the aforementioned drawbacks associated with state-of-the-art systems, by providing a sustainable, robust and relatively low cost setup and low maintenance system, enabling continuous production of biodiesel, allowing adding a desired amount of alcohol without causing damage to the enzyme activity, obviating the need to add water or aqueous buffer and preventing the wash away of the enzyme from the system.

### DESCRIPTION OF THE DRAWINGS

The present invention will be understood more comprehensively from the following detailed description taken in conjunction with the appended drawings in which:

FIG. 1 is a schematic isometric view of an embodiment of trough shaped reactor in accordance with the present invention;

FIG. 2 is a schematic cross-sectional isometric view of an embodiment of trough shaped reactor in accordance with the present invention;

FIG. 3 is a schematic cross-sectional side view of an embodiment of trough shaped reactor in accordance with the present invention;

FIG. 4 is a schematic cross-sectional isometric view of an embodiment of trough shaped reactor in accordance with the present invention, incorporating templates with immobilized catalyst;

FIG. 5 is a schematic cross-sectional side view of an embodiment of trough shaped reactor in accordance with the present invention, incorporating templates with immobilized catalyst;

FIG. 6 is a graph of conversion rates, in percent, plotted as a function of time, in hours, resulted in an exemplary process conducted in a trough shaped reactor in accordance with the present invention, incorporating templates with immobilized lipase catalyst;

FIG. 7 is a graph of conversion rates, in percent, plotted as a function of time, in hours, resulted in another exemplary process conducted in a trough shaped reactor, incorporating templates with immobilized lipase catalyst;

FIG. 8 is a graph of conversion rates, in percent, plotted as a function of time, in hours, resulted in yet another exemplary process conducted in a trough shaped reactor, incorporating templates with immobilized lipase catalyst;

FIG. 9 is a graph of conversion rates, in percent, plotted as a function of time, in hours, resulted in still another exemplary process conducted in a trough shaped reactor, incorporating templates with immobilized lipase catalyst;

FIG. 10 is a graph of conversion rates, in percent, plotted as a function of time, in hours, resulted in still yet another exemplary process conducted in a trough shaped reactor, incorporating templates with immobilized lipase catalyst;

FIG. 11 is a graph of conversion rates, in percent, plotted as a function of time, in hours, resulted in yet still another exemplary process conducted in a trough shaped reactor, incorporating templates with immobilized lipase catalyst;

FIG. 12 is a graph of conversion rates, in percent, plotted as a function of time, in hours, resulted in another exemplary process conducted in a trough shaped reactor, incorporating templates with immobilized lipase catalyst;

FIG. 13 is a graph of conversion rates, in percent, plotted as a function of time, in hours, resulted in another exemplary process conducted in a trough shaped reactor, incorporating templates with immobilized lipase catalyst.

### DETAILED DISCLOSURE OF EMBODIMENTS

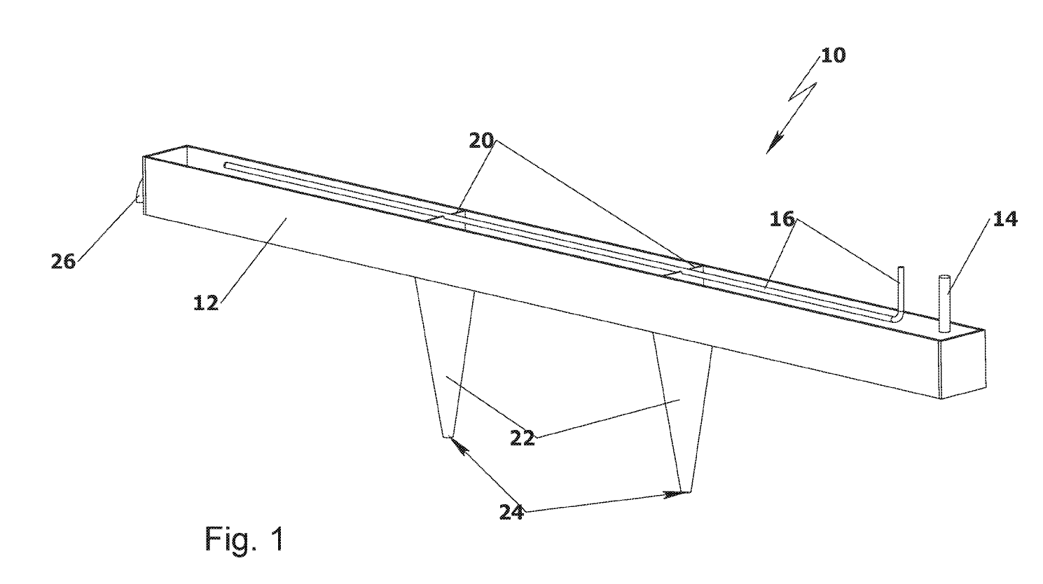

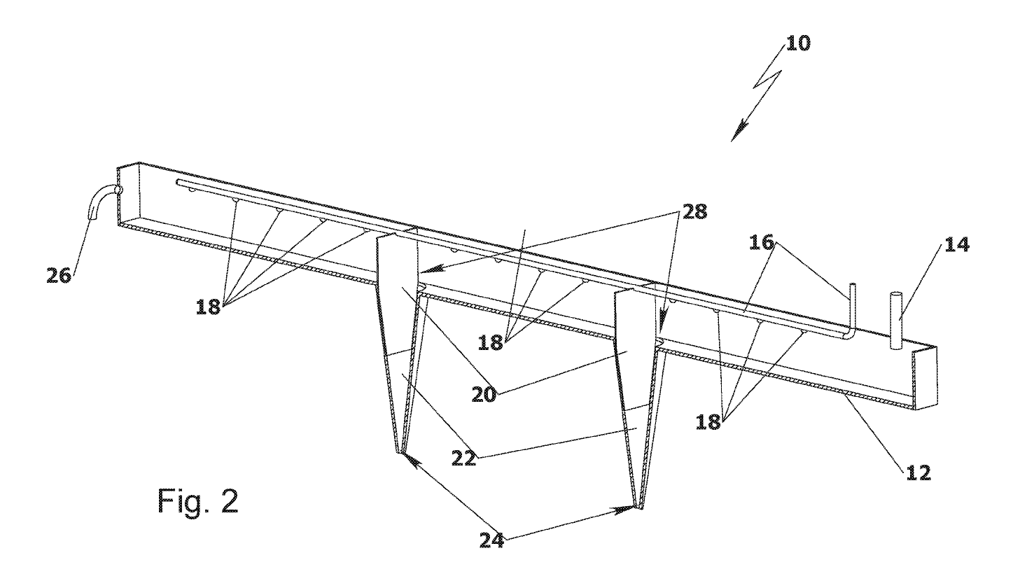

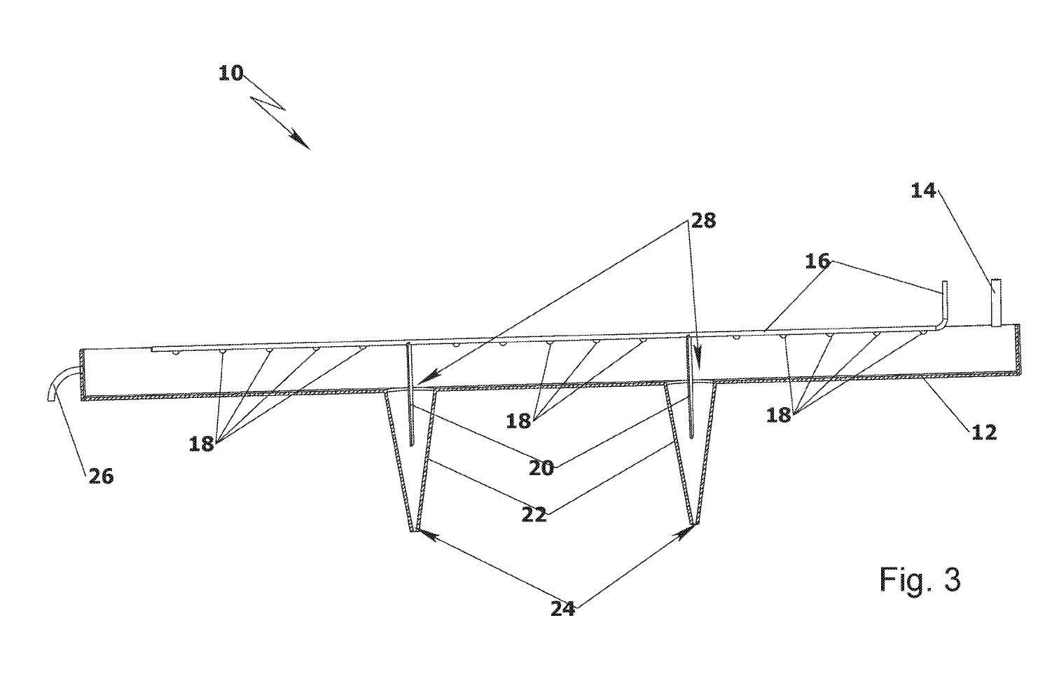

In accordance with some embodiments of the present invention, reference is now made to FIGS. 1 to 3, showing trough reactor 10. Trough reactor 10 comprises elongated trough shaped enclosure 12. Elongated trough shaped enclosure 12 is slightly inclined horizontally, to an angle ranging between about 1 degree and about 15 degrees; whereas a preferred inclination angle is about 2 degrees. The horizontal inclination of elongated trough shaped enclosure 12 at the aforementioned angle sustains a spontaneous gravitational flow along the lengths of trough reactor 10. Trough reactor 10 further comprises substrate stock feed inlet 14. Substrate stock feed inlet 14 is disposed at the upper terminal portion of elongated trough shaped enclosure 12. Substrate stock feed inlet 14 is configured to supply the substrate of the reaction conducted in trough reactor 10. Substrate stock feed inlet 14 is optionally comprises a means (not shown) for controlling the volumetric flow of the substrate into trough reactor 10, such as a baffle or valve (not shown). Trough reactor 10 further comprises reactant feed-in and distribution system 16. Feed-in and distribution system 16 is configured to supply the reactant of the reaction conducted in trough reactor 10. In some examples, reactant feed-in and distribution system 16 comprises a conduit extending along a substantial length of the upper portion of trough reactor 10. Feed-in and distribution system 16 preferably comprises a plurality of nozzles or sprinklers 18 disposed, typically equidistantly, on the conduit extending along the substantial length of the upper portion of trough reactor 10. Nozzles or sprinklers 18 disposed on the conduit of feed-in and distribution system 16 preferably configured to confer optimal spatial dispersal to the reactant across the surface of trough reactor 10. Feed-in and distribution system 16 is optionally comprises a means (not shown) for controlling the volumetric flow of the reactant into trough reactor 10 and/or efficient distribution thereof through nozzles or sprinklers 18, such as a baffle or valve (not shown).

Trough reactor 10 further comprises separation plates 20 disposed vertically in elongated trough shaped enclosure 12. Separation plates 20 obstruct the flow through elongated trough shaped enclosure 12. Separation plates 20 define mixing points 28 at the point of obstruction of the flow through elongated trough shaped enclosure 12 of reactor 10. At mixing points 28 along trough reactor 10 the substrate and/or reactant and/or product are mixed substantially homogenously to be further separated, as elaborated hereunder.

Trough reactor 10 further comprises separation columns 22. In the instance of trough reactor 10 separation columns 22 are conically shaped structures. It would be appreciated that conically shaped separation columns 22 of trough reactor 10 are merely exemplary; whereas any essentially hollow vertical structures are equally contemplated and applicable to trough reactor 10. Separation plates 20 extend downwardly underneath the bottom face of trough reactor 10 into separation columns 22 thereby forming a continuous vertical decanter structure, configured to separate a substrate and/or reactant and/or product from the mixture thereof, by the means of a continuous gravitational decantation process. Typically, the substrate and/or reactant and/or product is/are somewhat immiscible liquids, namely incapable of being mixed in various proportions to form a truly homogeneous solution. Accordingly, the substrate and/or reactant and/or product can be separated from a mixture thereof by the means of a continuous gravitational decantation process spontaneously occurring in separation columns 22 of trough reactor 10.

Separation columns 22 of trough reactor 10 terminate with outlets 24, at the bottom portion of separation columns 22. Outlets 24 of separation columns 22 are configured to drain an excessive portion of substrate and/or reactant and/or product from trough reactor 10. If the substrate or reactant has been removed from outlets 24 of separation columns 22, it is typically recycled by being eventually returned to substrate stock feed inlet 14 or reactant feed-in and distribution system 16, respectively.

Trough reactor 10 further comprises posterior outlet 26, configured to drain a portion of product and/or substrate and/or reactant from the interior of reactor 10. The essentially elongated shape of trough reactor 10 forming a moderate gravitation flow of the substrate from stock feed inlet 14 achieved by a slight inclination angle, in combination with a cascade of droplets or aerosols of the reactant produced by feed-in and distribution system 16, sustain optimal conditions for numerous reactions, as will be elaborated hereunder. Moreover, vertical decanter structures, formed by separation plates 20 extending underneath the bottom face of trough reactor 10 into separation columns 22 allowing continuously enriching the mixture along trough reactor 10 by withdrawing a selected fraction of substrate and/or reactant and/or product from outlets 24.

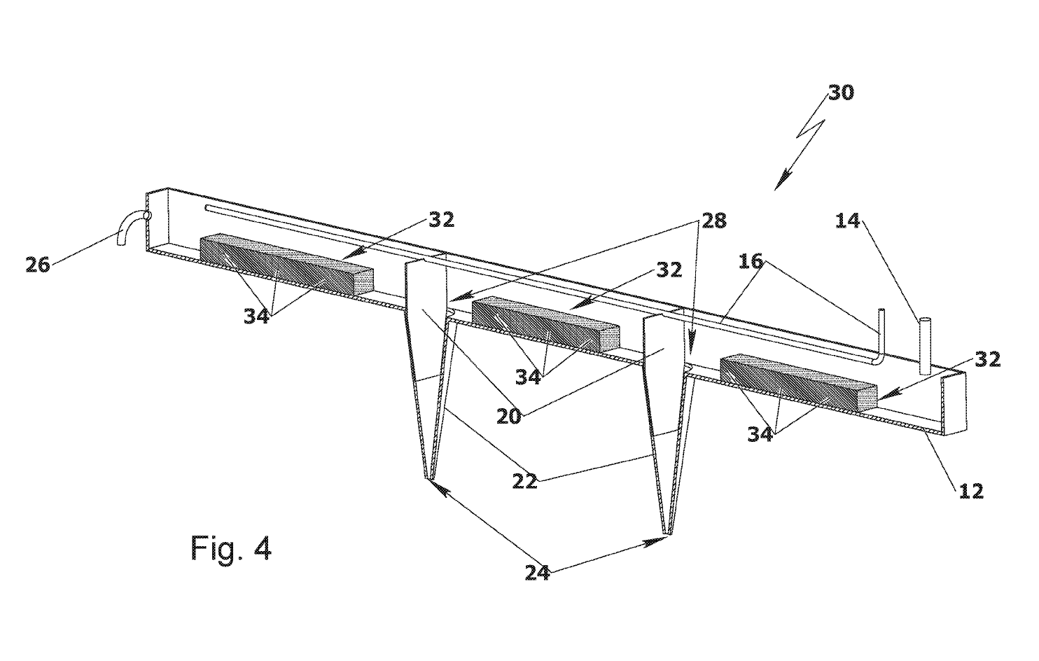

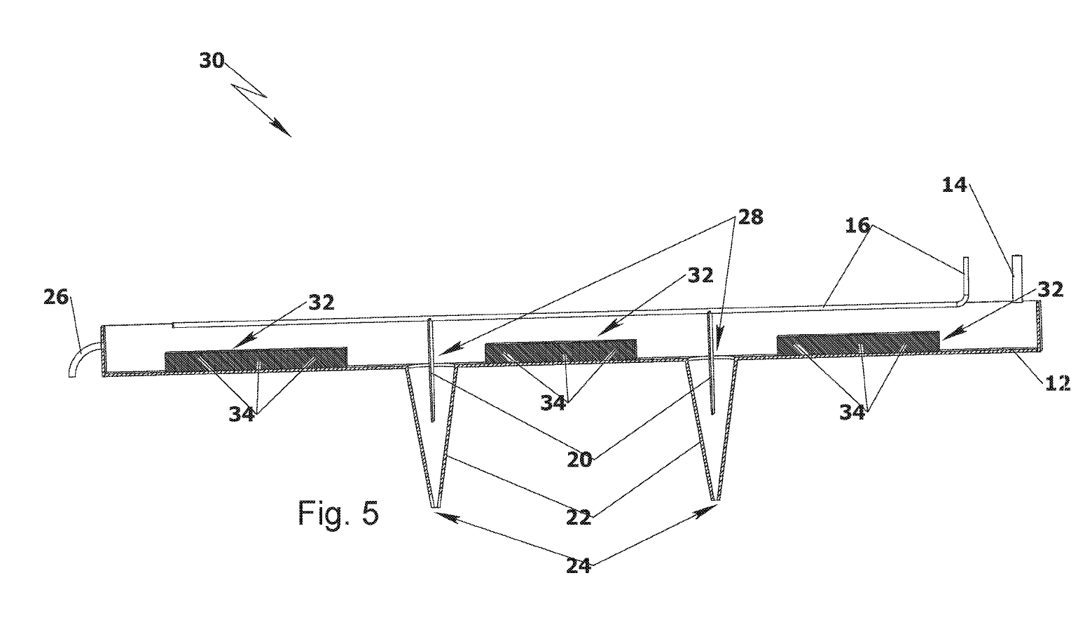

In accordance with some preferred embodiments reference is now made to FIGS. 4 and 5, showing trough reactor 30. Trough reactor 30 comprises templates 32, accommodating chemical or biochemical catalyst 34, connected to or embedded into the structure of templates 32. Templates 32 preferably embody a somewhat porous, membraneous or fibrous structure, characterized by a relatively large surface area, allowing the moderate gravitation flow of the substrate from stock feed inlet 14 to pass through, while concurrently allowing reactant to infiltrate from above and to intermix with the substrate, while being optimally exposed to the catalyst 34 on the vast surface area of templates 32. Examples of materials templates 32 are made of include any organic or inorganic material, in a non-limiting manner such as fiberglass, polyester membrane, membrane filters, cellulose filter papers, etc., with pore size ranging from about 0.01 micron to about 50 microns.

Templates 32 shown in FIGS. 4 and 5 are preferably adjoined to the inner bottom and inner side faces of trough reactor 30; thereby essentially obstructing a free flow through trough reactor 30 and forcing the moderate gravitation flow of the substrate from stock feed inlet 14 to pass through templates 32 matrix, while the substrate being optimally exposed to the catalyst 34 on the vast surface area of templates 32. Templates 32 are preferably removable and modularly replaceable as cartridges by new template cartridges (not shown), upon the reduction in efficacy of the catalyst 34 and/or for maintenance thereof.

### BEST MODE FOR PRACTICING AND CARRYING OUT THE INVENTION

In accordance with some preferred embodiments, a reaction for production of Methylester and Glycerol from Triacylglycerol and Methanol is performed in trough reactor of the present invention, in the presence of immobilized biochemical catalyst lipase, in accordance with Equation 1

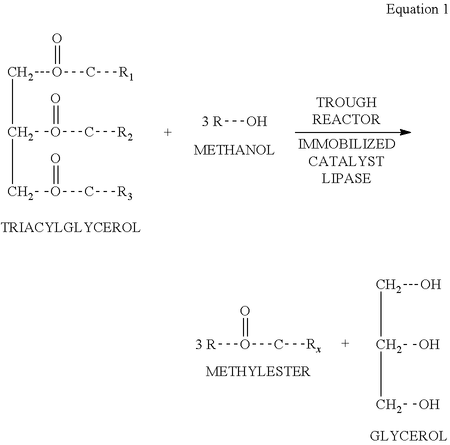

### Equation 1 Substrate TRIACYLGLYCEROL, Reactant METHANOL Product METHYLESTER and Co-product GLYCEROL

Methylester which is efficiently produced from the Triacylglycerol substrate and Methanol reactant in the trough reactor in the presence of lipase immobilized to the catalyst templates can be used as biodiesel fuel, virtually—without any other further processing. The embodiment where the trough reactor is implemented for biodiesel production in accordance with Equation 1, the undesired co-product or by-product of the reaction, namely glycerol, is separated spontaneously by gravitation in separation columns 22 of trough reactor 10 from the rest of the substrate and reactant as well as from the desired Methylester product. The undesired glycerol by-product is removed from outlets 24 of separation columns 22. The removal of the glycerol by-product from the system contributes to more efficient performance of the enzyme and thus increases the conversion rates and consequently the final concentration of the product. The preferred enzyme is any sn-1,3 positional specific lipase.

It should be acknowledged that the preferred instance of sn-1,3 positional specific lipase, in accordance with the preferred embodiment hereinabove, in a non-limiting manner includes: Thermomyces lanuginose, Rhizomucor miehei, Mucor miehei, Pseudomonas sp., Rhizopus sp., Mucor javanicus, Penicillium roqueforti, Aspergillus niger, Acromobacter sp. or Burkholderia sp. The lipase may have increased affinity for partial glycerides in a non-limiting manner including: Candida antarctica B, Candida rugosa, Alcaligenes sp. or Penicillium camembertii. Other lipases are equally contemplated within the scope of the preferred embodiment hereinabove, in a non-limiting manner including lipases derived from: Rhizopus niveus, Rhizopus oryzae, Burkholderia sp., Chromo-bacterium viscosum, papaya seeds or pancreatin. It should be acknowledged that the instance of the enzyme, in accordance with the preferred embodiment hereinabove, in a non-limiting manner includes: any region—specific or—unspecific lipase, phospholipase, esterase and alike, which may have been derived from any plant, animal, microorganism, such as: Chromobacterium viscasum, Cseudomonas spp, Cseudomonas fluorescens, Candida cuvata, Candida cylindracea, Aspergillus niger, Mucor miehe, Rhizopus arrizus.

### WORKING EXAMPLES

Example 1 In first empirical working example, the following materials and procedures were used to immobilize lipase enzyme to the catalyst templates, deployed in the trough reactor of the invention, for production of biodiesel in accordance with the method set forth hereinabove.

The method of immobilizing the lipase enzyme to the catalyst templates included three major steps. The first step involved preparation of an aqueous solution with predefined concentration of the enzyme lipase. The second step involved saturating a Hydrophilic Mixed Celluse Ester (MCE) membrane in the solution prepared at the first step and incubating therein for a while. The last major and third step involved drying the templates until an essentially minute or residual amount of water remained therein.

Exemplary first step included gradually adding 200 gr of raw Lipozome TL enzyme to a vessel containing 500 ml of distilled water, during continuous non-vigorous stirring. Lipozome TL enzyme was obtained from Novozymes at 77 Perry Chapel Church Road Franklinton, NC 27525 United States. The solution has then been stirred for 30 min at the temperature of 35 degrees Celsius. Then 0.1 percent (w/v) of Sodium Alginate, obtained from Sigma-Aldrich, CAS No. 9005-38-3, was added to the solution and the resulting mixture had been subsequently further stirred for 30 min at 35 degrees Celsius.

It should be acknowledged that any combination of enzymes, in various proportions, is equally applicable by creation of mixed enzyme solution during first step. Moreover, numerous adhesive agents are equally applicable in lieu of Sodium Alginate, to facilitate adherence of the enzyme to the template structure.

Exemplary second step included initially pouring the solution prepared at the first step into an open flat bottom vessel. The vessel was positioned horizontally so that the solution formed an essentially uniform layer of about 1 cm thickness. Thereafter a sheet of MCE Membrane Filter, obtained from Hangzhou ANOW® Microfiltration Corporation at Qingming Bridge, Xindeng Industrial Zone, Fuyang, Hangzhou, 311404 China, comprising Hydrophilic Mixed Celluse Ester (MCE) of 85-110 micron in thickness, having pore size of 0.1 to 5 micron, was cut up into pieces of 5 cm to 12 cm and arranged in layers piled up one on top of another to form a rectangular shaped template structure of several centimeters in height. Approximately 200 layers of 5 to 12 cm sized pieces of MCE Membrane Filter were arranged piled up one on top of another to form a rectangular template structure of about 3 centimeters in height. In total six rectangular template structure of about 3 centimeters in height were formed. A side portion of the rectangular shaped template structure was then submerged within the enzyme solution in the vessel open flat bottom vessel. The enzyme solution was then allowed to wick through the rectangular shaped template structure upwards, due to the spontaneous capillarity motion of the enzyme solution towards Hydrophilic Mixed Celluse Ester (MCE), until the entire structure of the template was essentially completely soaked up and saturated with the enzyme solution. The process of saturating the rectangular shaped template structure with enzyme solution has lasted approximately two hours.

Exemplary third step included drying the templates soaked up with enzyme solution, not in excessive temperature, until a minute or residual amount of water essentially not exceeding 8 present by weight remained in each template. The process of drying the templates previously soaked up with enzyme solution until sufficiently dry has lasted several hours.

Example 2 In second empirical working example, the following materials and procedures were used to produce biodiesel by catalyst templates with immobilized lipase enzyme, produced in accordance with Example 1, as set forth hereinabove, deployed in a rather miniature trough reactor, having the dimensions of: 80 cm in length, by 5 cm in width and 3 cm in height. The trough reactor was double-jacketed to maintain a constant temperature of operation. The reactant feed-in and distribution system included six sprinkling nozzles, two per each segment of the reactor, positioned essentially above the catalyst templates.

Catalyst templates with immobilized lipase enzyme, produced in accordance with Example 1, were positioned in the trough reactor, so that upon installation the totaling amount of the Lipozome TL, obtained from Novozyrnes USA, was about 182 gr. Different stoichiornetric ratios of methanol versus canola or palm oil, with and without addition of free fatty acids, at various flow rates, were tested in series of different manufacture conditions, elaborated infra. The reaction was conducted at the temperature of 32 degrees Celsius for durations of 10, 20, 30, 50, 70, 90, 120, 140, 160, 180 and 200 hours. The conversion of the raw materials was determined by measuring the percentage of alkyl esters in the final product.

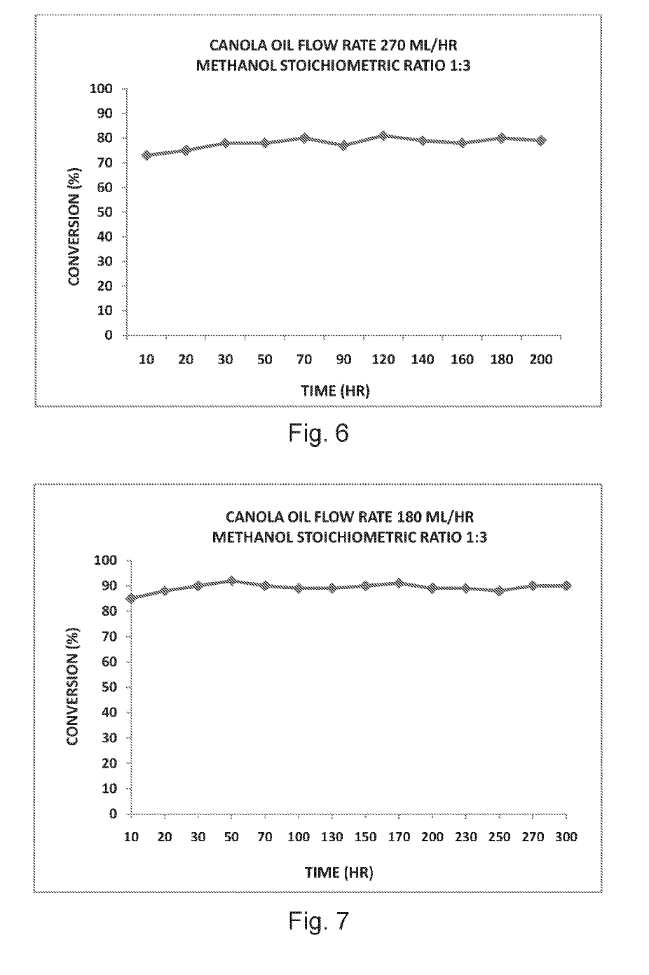

Example 3 Substrate flow rate of 270 ml of canola oil and reactant flow rate of 38.5 ml of methanol per 1 hi, at molecular ratio of 1 to 3, respectively, where supplied into the trough reactor, constructed in accordance with Example 2. The ratio of the flow rate to the amount of the enzyme was 1.5 and 1, respectively. The results of the conversion rates over time are provided in Table 1 infra and plotted in the graph in FIG. 6.

TABLE 1

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 73 | 75 | 78 | 78 | 80 | 77 | 81 | 79 | 78 | 80 | 79 |
| version | | | | | | | | | | | |
| (%) | | | | | | | | | | | |

Example 4 Substrate flow rate of 180 nil of canola oil and reactant flow rate of 25.5 ml of methanol per 1 hi, at molecular ratio of 1 to 3, respectively, where supplied into the trough reactor, constructed in accordance with Example 2. The ratio of the flow rate to the amount of the enzyme was 1 and 1, respectively. The results of the conversion rates over time are provided in Table 2 infra and plotted in the graph in FIG. 7.

TABLE 2

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 85 | 88 | 90 | 92 | 90 | 89 | 89 | 90 | 91 | 89 | 89 |
| version | | | | | | | | | | | |
| (%) | | | | | | | | | | | |

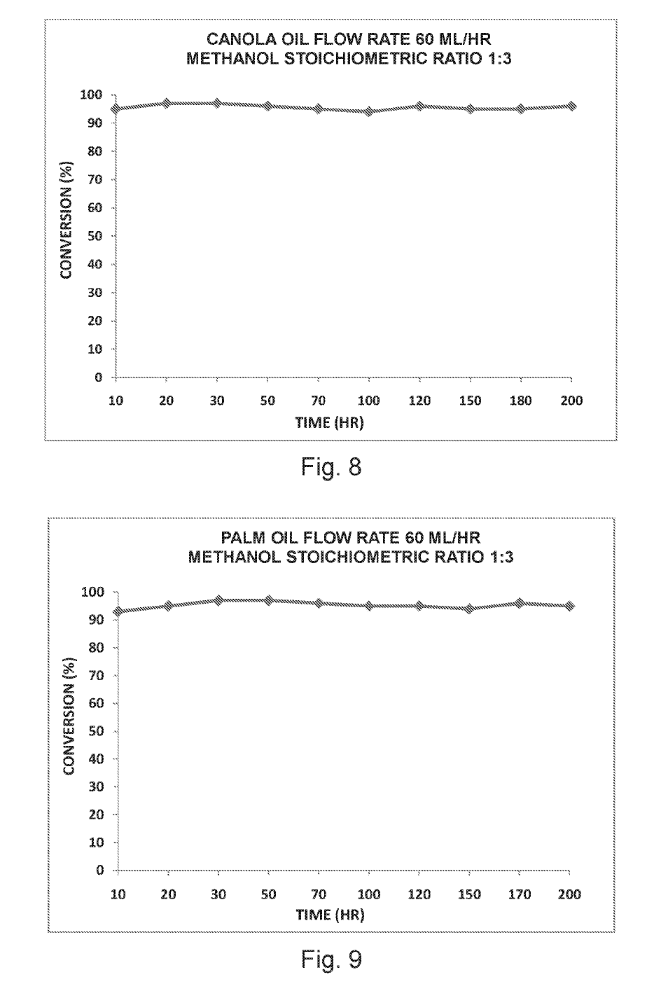

Example 5 Substrate flow rate of 60 ml of canola oil and reactant flow rate of 8.5 ml of methanol per 1 hr, at molecular ratio of 1 to 3, respectively, where supplied into the trough reactor, constructed in accordance with Example 2. The ratio of the flow rate to the amount of the enzyme was 0.33 and 1, respectively. The results of the conversion rates over time are provided in Table 3 infra and plotted in the graph in FIG. 8.

TABLE 3

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 95 | 97 | 97 | 96 | 95 | 94 | 96 | 95 | 95 | 96 | 95 |
| version | | | | | | | | | | | |
| (%) | | | | | | | | | | | |

Example 6 Substrate flow rate of 60 ml of palm oil and reactant flow rate of 8.5 ml of methanol per 1 hr, at molecular ratio of 1 to 3, respectively, where supplied into the trough reactor, constructed in accordance with Example 2. The ratio of the flow rate to the amount of the enzyme was 0.33 and 1, respectively. The results of the conversion rates over time are provided in Table 4 infra and plotted in the graph in FIG. 9.

TABLE 4

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 93 | 95 | 97 | 97 | 96 | 95 | 95 | 94 | 96 | 95 | 93 |
| version | | | | | | | | | | | |
| (%) | | | | | | | | | | | |

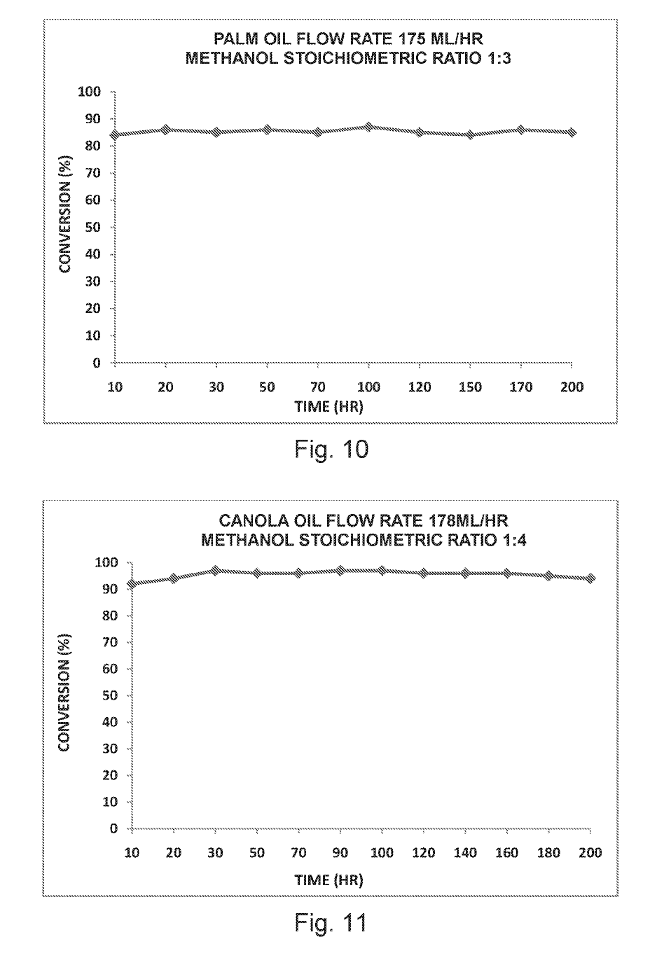

Example 7 Substrate flow rate of 175 ml of palm oil and reactant flow rate of 24.2 ml of methanol per 1 hr, at molecular ratio of I to 3, respectively, where supplied into the trough reactor, constructed in accordance with Example 2. The ratio of the flow rate to the amount of the enzyme was 1 and 1, respectively. The results of the conversion rates over time are provided in Table 5 infra and plotted in the graph in FIG. 10.

TABLE 5

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 84 | 86 | 85 | 86 | 85 | 87 | 85 | 84 | 86 | 85 | 84 |
| version | | | | | | | | | | | |
| (%) | | | | | | | | | | | |

Example 8 Substrate flow rate of 178 ml of canola oil and reactant flow rate of 32.7 ml of methanol per 1 hr, at molecular ratio of 1 to 4, respectively, where supplied into the trough reactor, constructed in accordance with Example 2. The ratio of the flow rate to the amount of the enzyme was 1 and 1, respectively. The results of the conversion rates over time are provided in Table 6 infra and plotted in the graph in FIG. 11

TABLE 6

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 92 | 94 | 97 | 96 | 96 | 97 | 97 | 96 | 96 | 96 | 95 |
| version | | | | | | | | | | | |
| (%) | | | | | | | | | | | |

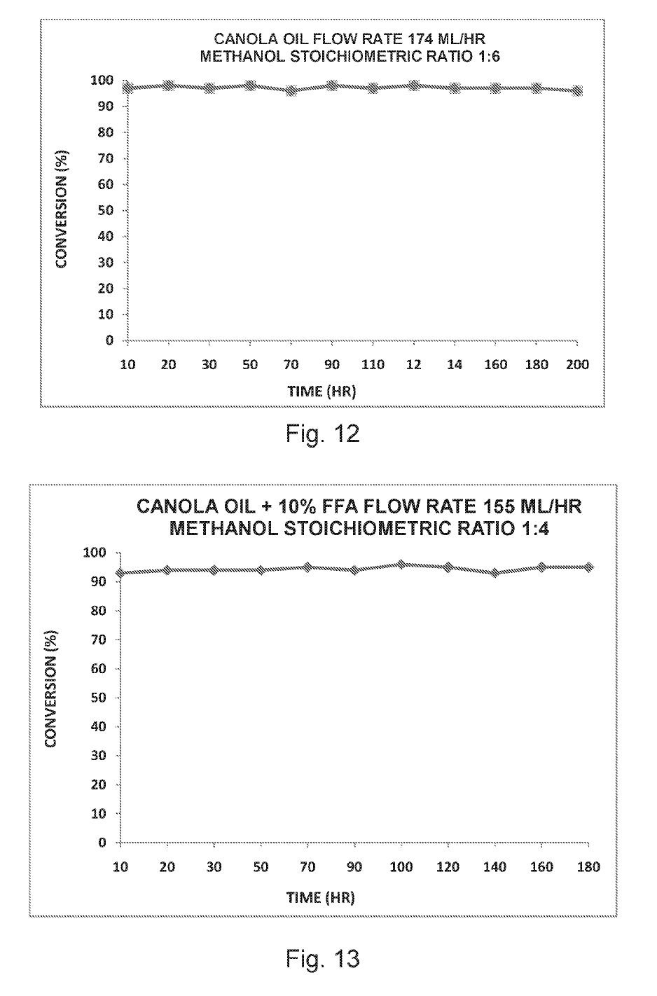

Example 9 Substrate flow rate of 175 ml of canola oil and reactant flow rate of 42.8 ml of methanol per 1 hr, at molecular ratio of 1 to 6, respectively, where supplied into the trough reactor, constructed in accordance with Example 2. The ratio of the flow rate to the amount of the enzyme was 1 and 1, respectively. The results of the conversion rates over time are provided in Table 7 infra and plotted in the graph in FIG. 12.

TABLE 7

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 97 | 98 | 97 | 98 | 96 | 98 | 97 | 98 | 97 | 97 | 97 |
| version | | | | | | | | | | | |
| (%) | | | | | | | | | | | |

Example 10 Substrate flow rate of 155 ml of canola oil that included 10% free fatty acids (FFA) and reactant flow rate of 29.1 ml of methanol per 1 hr, at molecular ratio of 1 to 4, respectively, where supplied into the trough reactor, constructed in accordance with Example 2. The ratio of the flow rate to the amount of the enzyme was 1 and 1, respectively. The results of the conversion rates over time are provided in Table 8 infra and plotted in the graph in FIG. 13.

TABLE 8

| | Time (hr) | | | | | | | | | | |
|---|---|---|---|---|---|---|---|---|---|---|---|
| | 10 | 20 | 30 | 50 | 70 | 90 | 120 | 140 | 160 | 180 | 200 |
| Con- | 93 | 94 | 94 | 94 | 95 | 94 | 96 | 95 | 93 | 95 | 95 |
| version | | | | | | | | | | | |
| (%) | | | | | | | | | | | |

1. A trough reactor configured to catalyze a reaction between triacylglycerol and methanol to produce methylester and glycerol, said trough reactor comprises:
(a) an elongated trough shaped enclosure, wherein said elongated trough shaped enclosure being horizontally inclined at an angle sustaining a spontaneous gravitational flow along said elongated trough shaped enclosure;
(b) a substrate stock feed inlet disposed at an upper terminal portion of said elongated trough shaped enclosure, said substrate stock feed inlet is configured to supply said a feed of said thacylglycerol for said reaction;
(c) a reactant feed-in and distribution system configured to supply and distribute a feed-in of said methanol for said reaction, said reactant feed-in and distribution system comprises:
(I) a conduit extending along a substantial length of un upper portion of said elongated trough shaped enclosure;
(II) a plurality of nozzles disposed on said conduit configured to confer optimal spatial dispersal of said methanol within said elongated trough shaped enclosure;
(d) at least one separation plate disposed essentially vertically in said elongated trough shaped enclosure, said at least one separation plate extends downwardly underneath a bottom face of said elongated trough shaped enclosure, wherein said at least one separation plate is configured to substantially obstruct said spontaneous gravitational flow along said elongated trough shaped enclosure;
(e) at least one mixing point defined along said trough reactor, at an intersection of said at least one separation plate with said elongated trough shaped enclosure; wherein at least two members selected from the group consisting of: said triacylglycerol, said methanol, said methylester and said glycerol, are mixed in said at least one mixing point into a mixture;
(f) at least one separation column comprising an essentially hollow vertical structure, wherein said at least one separation plate extends downwardly underneath said bottom face of said elongated trough shaped enclosure, into said essentially hollow vertical structure of said at least one separation column;
(g) wherein said at least one separation column in combination with said at least one separation plate forming a continuous gravitational decanter, configured to separate at least one member selected from the group consisting of: said triacylglycerol, said methanol, said methylester and said glycerol, from said mixture, by a means of continuous gravitational decantati on process;
(h) at least one outlet, at a bottom portion of said at least one separation column, configured to drain an excessive portion of at least one member selected from the group consisting of: said triacylglycerol, said methanol, said methylester and said glycerol, separated from said mixture in said continuous gravitational decanter by said continuous gravitational decantation process;
(i) a posterior outlet, disposed at a posterior end of said elongated trough shaped enclosure, configured to drain a portion of at least one member selected from the group consisting of: said triacylglycerol, said methanol, said methylester and said glycerol, from said trough reactor;
(j) at least one template comprising a matrix selected from the group consisting of: a porous matrix, membraneous matrix and fibrous matrix, characterized by a large surface area; said at least one template configured to essentially obstruct said spontaneous gravitational flow along said elongated trough shaped enclosure, thereby forcing said spontaneous gravitational flow to pass through said at least one template matrix, while concurrently allowing said methanol to infiltrate from above and to intermix with said spontaneous gravitational flow;
(k) a catalyst selected from the group consisting of: a chemical catalyst and biochemical catalyst, said catalyst being affixed to said at least one template matrix.

2. The trough reactor as set forth in claim 1, wherein said angle sustaining a spontaneous gravitational flow is selected from the group consisting of: an angle ranging between 1 degree and 15 degrees and angle of 2 degrees.

3. The trough reactor as set forth in claim 1, wherein said at least one template comprises a material selected from the group consisting of: fiberglass, polyester membrane, membrane filters, cellulose filter and paper filter.

4. (canceled)

5. The trough reactor as set forth in claim 1, wherein said at least one outlet, at said bottom portion of said at least one separation column, is configured to drain an excessive portion of said triacylglycerol or said methanol; wherein said triacylglycerol or said methanol drained from said outlet of said at least one separation column is recycled into said substrate stock feed inlet or said reactant feed-in and distribution system.

6. The trough reactor as set forth in claim 1, wherein said catalyst is a lipase enzyme.

7. The trough reactor as set forth in claim 1, wherein said catalyst is selected from the group consisting of: a sn-1,3 positional specific lipase, Thermomyces lanuginosa lipase, Rhizomucor miehei lipase, Mucor miehei lipase, Pseudomonas species lipase, Rhizopus species lipase, Mucor javanicus lipase, Penicillium roqueforti lipase, Aspergillus niger lipase, Acromobacter species Lipase, Burkholderia species Lipase, Candida antarctica B lipase, Candida rugosa lipase, Alcaligenes species lipase, Penicillium camembertii lipase, Rhizopus niveus lipase, Rhizopus oryzae lipase, Burkholderia species lipase, Chromo-bacterium viscosum lipase, papaya seeds lipase, pancreatin lipase, Chromobacterium viscasum lipase, Cseudomonas species lipase, Cseudomonas fluorescens lipase, Candida cuvata lipase, Candida cylindracea lipase, Mucor miehe lipase, Rhizopus arrizus lipase.

8. (canceled)

9. (canceled)

10. (canceled)

11. (canceled)

12. (canceled)

13. (canceled)

14. A gravitational trough reactor configured to catalyze a reaction between a substrate and a reactant to produce at least one product, said trough reactor comprises:
(a) an elongated trough shaped enclosure, wherein said elongated trough shaped enclosure being horizontally inclined at an angle sustaining a spontaneous gravitational flow along said elongated trough shaped enclosure;
(b) a substrate stock feed inlet disposed at an upper terminal portion of said elongated trough shaped enclosure, said substrate stock feed inlet is configured to supply a feed of said substrate into said elongated trough shaped enclosure;
(c) a reactant feed-in and distribution system configured to supply and distribute a feed-in of said reactant across said elongated trough shaped enclosure;
(d) at least one separation plate disposed essentially vertically in said elongated trough shaped enclosure, said at least one separation plate extends downwardly underneath a bottom face of said elongated trough shaped enclosure, wherein said at least one separation plate is configured to substantially obstruct said spontaneous gravitational flow along said elongated trough shaped enclosure;
(e) at least one separation column comprising an essentially hollow vertical structure, wherein said at least one separation plate extends downwardly underneath said bottom face of said elongated trough shaped enclosure, into said essentially hollow vertical structure of said at least one separation column;
wherein said at least one separation column in combination with said at least one separation plate forming a continuous gravitational decanter, configured to separate at least one member selected from the group consisting of: said substrate, said reactant and said at least one product, from said mixture, by a means of continuous gravitational decantation process;
(f) at least one outlet, at a bottom portion of said at least one separation column, configured to drain an excessive portion of at least one member selected from the group consisting of: said substrate, said reactant and said at least one product, separated in said continuous gravitational decanter by said continuous gravitational decantation process;
(g) a posterior outlet, disposed at a posterior end of said elongated trough shaped enclosure, configured to drain a portion of at least one member selected from the group consisting of: said substrate, said reactant and said at least one product, from said elongated trough shaped enclosure.

15. The trough reactor, as set forth in claim 14, further comprises at least one template comprising a matrix selected from the group consisting of:
a porous matrix, membraneous matrix and fibrous matrix, characterized by a large surface area; wherein said at least one template configured to essentially obstruct said spontaneous gravitational flow along said elongated trough shaped enclosure, thereby forcing said spontaneous gravitational flow to pass through said at least one template matrix, while concurrently allowing said reactant to infiltrate from above and to intermix with said spontaneous gravitational flow.

16. The trough reactor, as set forth in claim 14, further comprises a catalyst selected from the group consisting of: a chemical catalyst and biochemical catalyst, said catalyst being affixed to said at least one template matrix.

17. The trough reactor as set forth in claim 14, wherein as at least one mixing point is defined along said trough reactor, at an intersection of said at least one separation plate with said elongated trough shaped enclosure;
wherein at least two members selected from the group consisting of: said substrate, said reactant and said at least one product, are mixed into a mixture.

18. The trough reactor as set forth in any one of the claim 14, wherein said reactant feed-in and distribution system comprises:
(a) a conduit extending along a substantial length of un upper portion of said elongated trough shaped enclosure;
(b) a plurality of nozzles disposed on said conduit configured to confer optimal spatial dispersal of said methanol within said elongated trough shaped enclosure.

19. A method of catalyzing a reaction between a substrate and reactant to produce at least one product, in a trough reactor, said method comprises:
(a) providing an elongated trough shaped enclosure;
(b) horizontally inclining said elongated trough shaped enclosure at an angle sustaining a spontaneous gravitational flow along said elongated trough shaped enclosure;
(c) supplying a feed of said substrate into said elongated trough shaped enclosure;
(d) supplying and distributing a feed-in of said reactant into said elongated trough shaped enclosure;
(e) disposing at least one separation plate essentially vertically in said elongated trough shaped enclosure, extending downwardly underneath a bottom face of said elongated trough shaped enclosure;
(f) substantially obstructing said spontaneous gravitational flow along said elongated trough shaped enclosure by said at least one separation plate;
(g) providing at least one separation column comprising an essentially hollow vertical structure and extending said at least one separation plate downwardly underneath said bottom face of said elongated trough shaped enclosure, into said essentially hollow vertical structure of said at least one separation column;
(h) separating at least one member selected from the group consisting of: said substrate, said reactant and said at least one product, by a means of continuous gravitational decantation process conducted in said at least one separation column;
(i) draining from an outlet at a bottom portion of said at least one separation column an excessive portion of at least one member selected from the group consisting of: said substrate, said reactant and said at least one product, in said at least one separation column by said continuous gravitational decantation process;
(j) draining a portion of at least one member selected from the group consisting of: said substrate, said reactant and said at least one product, from said trough reactor from a posterior outlet.

20. The method, as set forth in claim 19, further comprises disposing at least one template comprising a matrix selected from the group consisting of: a porous matrix, membraneous matrix and fibrous matrix, characterized by a large surface area, in said elongated trough shaped enclosure.

21. The method, as set forth in claim 20, further comprises affixing a catalyst selected from the group consisting of: a chemical catalyst and biochemical catalyst, to said at least one template matrix.

22. The method, as set forth in claim 20, further comprises essentially obstructing said spontaneous gravitational flow along said elongated trough shaped enclosure, by said at least one template, thereby forcing said spontaneous gravitational flow to pass through said at least one template matrix.

23. (canceled)

24. The method, as set forth in claim 19, further comprises mixing at least two members selected from the group consisting of: said substrate, said reactant and said at least one product, into a mixture in at least one mixing point defined along said trough reactor, at an intersection of said at least one separation plate with said elongated trough shaped enclosure.

25. The method as set forth in claim 20, wherein said at least one template comprises a material selected from the group consisting of: fiberglass, polyester membrane, membrane filters, cellulose filter and paper filter.

26. (canceled)

27. The method as set forth in claim 19, wherein said draining of said excessive portion from said outlet at said bottom portion of said at least one separation column is draining said substrate or said reactant; wherein said substrate or said reactant is recycled into said feed of or said feed-in of said methanol.

28. The method as set forth in claim 21, wherein said substrate is triacylglycerol, said reactant is methanol, said at least one product is methylester and said glycerol and said catalyst is a lipase enzyme.

29. The method as set forth in claim 21, wherein affixing of said catalyst comprises: (a) preparing an aqueous solution with enzyme lipase; (b) saturating said template comprising hydrophilic mixed cause ester membrane with said solution and incubating said template saturated with said solution; (c) drying said template until an essentially minute residual amount of water remains therein.
